(19) United States
(12) Patent Application Publication
(10) Pub. No.: US 2004/0072765 A1
(43) Pub. Date: Apr. 15, 2004

(54) Cardiovascular and bone treatment using isoflavones

(75) Inventors: Graham Edmund Kelly, Northbridge (AU); Alan James Husband, McMahons Point (AU)

Correspondence Address: FINNEGAN, HENDERSON, FARABOW, GARRETT & DUNNER, LLP, 1300 I STREET, NW, WASHINGTON, DC, 20005, US

(73) Assignee: Novogen Research Pty Ltd.

(21) Appl. No.: 10/600,004

(22) Filed: Jun. 18, 2003

Related U.S. Application Data
(63) Continuation of application No. 09/914,035, filed on Dec. 10, 2001, now abandoned.
Application No. 09/914,035 is a 371 of international application No. PCT/AU00/00384, filed on Apr. 27, 2000.

(30) Foreign Application Priority Data
Apr. 28, 1999 (AU) PQ 0083

(51) Int. Cl. A61K 31/7048; A61K 31/353

(52) U.S. Cl. 514/27; 514/456

(57) ABSTRACT

Compositions comprising formononetin and/or one or more isoflavones selected from biochanin, genistein and daidzein, in a therapeutically effective ratio of formononetin to said isoflavones of 15:1 to 2:1, optionally in association with one or more carriers, excipients, auxiliaries and/or diluents are described. Also described are methods of treatment involving such compositions including the prevention and/or treatment of cardiovascular disease, the beneficial alteration of blood lipoprotein levels, or a reduction in the risk of vascular disease, or a reduction in the risk of coronary heart disease, or a reduction in the risk of arteriosclerosis, or in the beneficial alteration or maintenance of bone density such as in the prevention or treatment of osteoporosis, and/or in the prevention and/or treatment of bone fracture.

### FIELD OF THE INVENTION

The present invention relates to the treatment and/or prevention of cardiovascular diseases and osteoporosis using isoflavone compounds. More particularly it relates to compositions, uses and methods involving certain plant isoflavones, and even more particularly to compositions with high formononetin content, in the prevention and/or treatment of cardiovascular disease, or the beneficial alteration of blood lipoprotein levels, or to reduce the risk of coronary heart disease, or to reduce the risk of arteriosclerosis, or in the beneficial alteration or maintenance of bone density such as in the prevention or treatment of osteoporosis, and/or in the prevention and/or treatment of bone fractures.

### BACKGROUND OF THE INVENTION

Note: References are collected at the end of the description.

Cardiovascular disease and osteoporosis have emerged as major community health issues in Western communities that are experiencing increasing longevity and in non-Western communities that are progressively westernising their lifestyles, particularly diet. Current therapeutic and preventative options for both diseases are less than satisfactory, with current options either targeting specific symptoms and failing to address the underlying pathogenic mechanisms or being associated with dose-limiting undesirable side-effects. There is an urgent need to develop safer, more effective therapies that are directed at the underlying biological events that cause cardiovascular disease and osteoporosis and which could be used both to treat existing disease states and to prevent the onset of disease, and which could be used on a long-term basis without adverse consequences.

The primary cause of cardiovascular disease is a disease of artery walls known as atherosclerosis. Atherosclerosis is characterised by the deposition of fatty plaque within the walls of blood vessels and a resulting inflammatory process induced by that plaque. The consequence of this event is a thickening of the wall with a resulting diminution of the internal volume of the artery lumen. This consequence has two principal outcomes—(a) restricted blood supply to an end-organ, usually the heart (causing coronary heart disease), or kidney (causing renal failure) or the brain (causing senile dementia); and (b) acute cerebral ischaemia (or ‘stroke’) due to a piece of atheromatous plaque breaking free and travelling as an embolus until it lodges in a small diameter vessel resulting in injury to the area of tissue supplied by that vessel.

An important predisposing factor to the development of atherosclerosis is the level of cholesterol in the blood, or more specifically the form in which cholesterol is present in the blood. Cholesterol is an important cell structural component of cells and is required by most cells on a daily basis. Cholesterol is delivered to cells via the blood by being bound to a protein known as apoprotein of which there are several different types. The combination of cholesterol and apoprotein forms a particle known as lipoprotein. Cholesterol is delivered to cells in a particle known as low density lipoprotein (abbreviated to ‘LDL’) comprising a particular type of apoprotein attached to a small number of cholesterol molecules. In the tissues, the cholesterol is detached from its carrier apoprotein and used by the cell. Any excess cholesterol sits in a free form in the tissues until being collected by another type of apoprotein. This cholesterol is returned to the liver for recycling in the form of a particle known as high density lipoprotein (abbreviated to ‘HDL’). In healthy individuals, the ratio of LDL to HDL is in the range of about 2:1 to 2.5:1. It is believed generally that at this ratio, excess cholesterol, is unlikely to build up in the tissues. As this ratio increases, so the ability of the body to recycle excess cholesterol diminishes, leaving free cholesterol in tissues such as artery walls. Free cholesterol, particularly in artery walls, is prone to oxidation. Oxidised cholesterol is highly irritable, leading to inflammation in surrounding tissues. Atheromatous plaque is a combination of accumulating oxidised cholesterol and inflammatory tissue.

An increase in the LDL:HDL ratio above 3:1 generally is thought to be associated with increased risk of atherosclerosis. A large proportion of individuals in Western communities have a ratio of about 3:1 to 7:1. Aside from individuals with a familial predisposition to this problem, the principal causes of this imbalance are lifestyle factors and age. It is well known that an imbalance can be due to either an abnormally elevated LDL level, or an abnormally low HDL level, or both. Factors known to be associated with an elevated LDL level are mainly dietary, e.g. a diet comprising high levels of animal fat and low levels of complex carbohydrates. Factors known to be associated with a low HDL level are lack of exercise and advancing age. The age-related effect on HDL levels is a major contributor to a high LDL:HDL ratio in older people, particularly women because HDL production is related to estrogen levels in the body and estrogen levels decline in both women and men with advancing age.

While total blood cholesterol levels are thought to be a relevant risk factor for atherosclerosis, it is now generally believed that the normal total blood cholesterol range is very wide and that a more relevant risk factor is the LDL:HDL ratio. That is, in cardiovascular risk terms, the absolute levels of both lipoprotein types is subordinate to the relative proportion of LDL and HDL.

It can be seen that in a person with an abnormally high LDL:HDL ratio, a normal ratio of about 2.5:1 might be restored by a therapeutic strategy that either lowered the LDL level, or elevated the HDL level, or both. Current therapeutic options predominantly aim to lower the LDL level and three broad approaches are used. The first approach is the use of drugs that interfere with cholesterol synthesis. The so-called ‘statins’, for example ethyl-2-(p-chloropbenoxy)-2-methyl-propionate, reduce cholesterol levels in the blood by interrupting cholesterol biosynthesis in the liver. These drugs typically result in a decrease in blood LDL levels by between about 10-40%. The second approach is to reduce cholesterol absorption from the gut, thereby reducing the pool of cholesterol available within the body. Historically this has been through the use of binding agents, such as insoluble, high molecular weight polymers which bind to bile acids forming a complex that is excreted in the faeces. More recently, plant sterols have been found to achieve the same result. The increased faecal loss of bile acids with either material leads to a decrease in LDL levels, typically in the range 5-12%. The third approach involves the use of soy protein which typically reduces total cholesterol and LDL levels by about 8-12%. The mechanism of action of this material is unknown. There are a number of deficiencies with this approach focused on lowering LDL levels. The first is that the link between LDL-lowering and reduced risk of atherosclerosis or cardiovascular disease is assumed, but there is no firm clinical evidence to support this assumption. The second is that most of the current therapeutics are associated with undesirable side-effects. The statin drugs are associated with a high incidence of adverse side-effects including nausea, gastrointestinal reactions such as vomiting, loose stools, dyspepsia, abdominal distress, cardiovascular complications such as increased angina or cardiac arrhythmias, dermatological problems, plus various other general complications. The resin products produce adverse reactions such as gastrointestinal disturbance, constipation, aggravation of haemorrhoids, and abdominal discomfort.

An alternative therapeutic option is to elevate the HDL levels. This option is increasingly being regarded by the medical profession as the more desirable option for several reasons. First, because the most conclusive evidence for a clinical benefit resulting from a re-adjustment in the LDL:HDL ratio lies with the strategy of increasing the HDL level. Gordon et al (1989) (Circulation 79: 8-15) have shown that for every 1 mg/100 mg (1%) rise in HDL cholesterol in the blood, the risk of death from coronary heart disease decreases by 3%. Second, because HDL appears to provide beneficial actions on the artery wall beyond that of scavenging oxidised cholesterol. Third, because in older women in particular, the primary reason for an abnormally high LDL:HDL ratio is a decline in HDL levels. The therapeutic options here are more limited compared to those targeting LDL levels. The most effective therapy is steroidal estrogen such as estradiol. Estradiol or estrogen replacement therapy typically increases HDL levels by between about 15-30% in post-menopausal women, with little or no effect on LDL levels. However, estrogen replacement therapy is associated with a number of adverse cardiovascular outcomes including a predisposition to thrombogenesis, leading to increased risk of blood clots and stroke. This makes estrogen replacement therapy an unattractive therapeutic option for older women. Also, the feminising effects of estrogen make it even more unattractive as an option for men. Another drug substance known as clofibrinate will increase HDL levels in men and women by about 10% but is little used because of its adverse side-effects. Given that the most conclusive clinical evidence for a beneficial effect on atherosclerosis resulting from moderation of cholesterol levels comes from elevation of HDL levels, the current inability of medicine to offer a safe, effective means of achieving this outcome remains a major challenge.

Loss of bone density, like cardiovascular disease, is emerging as a major community health problem in Western communities that are experiencing increasing longevity. As with declining HDL levels with advancing age, loss of bone density appears to be associated primarily with declining estrogen levels in the body. One of the biological effects of estrogen is the stimulation of osteoblasts, those bone cells that are responsible for the production of new bone, and the down-regulation of osteoclasts, those bone cells responsible for the resorption of old bone. In the presence of low estrogen levels, osteoblast activity diminishes while osteoclast activity continues, leading to reduced production of new bone to replace the removed older bone. The result is a gradual loss of bone mass. The early stage of this condition is known as osteopenia. The later stage is known as osteoporosis. In osteoporosis the density of the bones has fallen to the point where they are liable to fracture.

There are two major types of bone tissue in the body -trabecular bone and cortical bone.* Trabecular bone accounts for about 80% of the bone in the body and is low density, areolar bone. Trabecular bone predominates in those bones or in those parts of bones which are not highly weight-bearing, such as the vertebrae, ribs, skull, wrist and ankle. Cortical accounts for the remaining 20% of bone in the body and is very dense bone. Cortical predominates in load-bearing situations such as the long bones of the limbs and the femoral neck in the hip joint.

Bone density begins to decline normally from about middle age in both men and women and both trabecular and cortical bone are affected, although trabecular bone has a higher natural turn-over rate compared to cortical bone, and trabecular bone typically experiences a greater rate of loss of density in the early part of this osteopenic process. Menopause accelerates this process in women. Near menopause, trabecular bone has about an eightfold greater rate of turnover compared to cortical bone. In peri-menopausal women, trabecular bone is lost by between about 4-8% per annum versus 2-3% per annum for cortical bone. By about the age of 60, the rates of loss in both trabecular and cortical bone approximate. This age-related effect is responsible for the phenomenon in menopausal women where fractures seen between the ages of 50-60 years typically involve the vertebrae, wrist and ribs (predominantly trabecular bone) and over the age of 60 years typically involve the hip and long bones (predominantly cortical bone). Hip and femur fracture are the most serious of the various bone fractures, requiring extended hospitalisation and usually extensive surgery. About one-third of older women who fracture their hip die within 12 months of the fracture because of related complications.

There are various therapeutic options for the treatment or prevention of osteoporosis. Steroidal estrogens such as estradiol or synthetic derivatives such as raloxifene are well known and widely used for these purposes. These compounds function through a combination of promotion of bone deposition and reduction in bone resorption. However, their effect is seen principally on trabecular bone and they have little or no effect on cortical bone. The result of this selective action is that they may protect against fracture of bones such as the wrist, ribs and vertebrae, but provide little or no protection against the more serious hip and femur fractures. A class of compounds known as bisphosphonates also enjoy common usage. These compounds act by decreasing bone resorption, and while affecting both trabecular and cortical bone, like the estrogens, their effect is predominantly directed towards trabecular bone. Other therapies include calcitonin, which decreases the rate of bone resorption and ipriflavone which inhibits bone resorption and increases osteoblast function. All of these drugs are associated with undesirable side-effects. Given that effective therapy or prevention of bone fractures requires long-term therapy of between 10-30 years, safety and tolerability are key issues for patients and all the above therapies enjoy poor patient compliance because of their low safety profiles. There is an urgent need to develop therapies that are particularly directed to protection of cortical bone and which have a high safety and tolerability profile so as to encourage long-term usage.

Some interest has been shown in recent years in plant compounds known as isoflavones, in particular those with estrogenic function such as genistein and daidzein and their methyl esters, biochanin and formononetin. In part this interest stems from the epidemiological observations that cardiovascular diseases and osteoporosis are less common in communities whose diets are rich in isoflavones. In part, it also stems from their estrogenic function and the likelihood that they could mimic the health benefits of estradiol, in particular in the positive cardiovascular health benefits and bone density-raising effects of estradiol. Most scientific interest has focused on genistein and daidzein as these are the strongest estrogen agonists of the four isoflavones. Genistein and daidzein are reported to have an estrogenic potency approximately 0.1% that of estradiol, while formononetin and biochanin are about 10-100× weaker than that.

The literature is minimal in respect to osteoporosis and isoflavone studies. U.S. Pat. No. 5,424,331 discloses the use of genistein and daidzein as components of an extensive mixture of specified compounds in the prevention and/or treatment of osteoporosis in humans. However, that patent does not teach the beneficial use of the isoflavones formononetin and biochanin, or the effect of a particular isoflavone ratio, or the beneficial effect of isoflavones alone, or the relative effect of the isoflavones either alone or in combination with other materials on trabecular or cortical bone.

There is experimental evidence that genistein has a beneficial effect on bone. Genistein is reported to stimulate bone formation (Fanti, 1998) and to depress osteoclast activity (U.S. Pat. No. 5,506,211 Barnes, S and Blair, H C: Genistein for use in inhibiting osteoclasts). Low doses of genistein reportedly increased both cortical and trabecular bone density in rats (Anderson, 1998). While the group of isoflavones, genistein, daidzein, formononetin and biochanin are known to share some biological properties, it is also well known that they vary considerably in their biological potencies. Thus there is no understanding of the effect of formononetin, biochanin or daidzein on bone biology.

The only reported clinical study involving isoflavones and osteoporosis involved 66 post-menopausal women in a placebo-controlled study who were treated for 6 months with a soy product containing either ‘moderate’ or ‘high’ isoflavone levels (Potter S. M. et al “Soy protein and isoflavones: their effects on blood lipids and bone density in postmenopausal women” American Journal of Clinical Nutrition 1998: 68(suppl) 1375S-1379S). It is well known that soy contains daidzein and genistein approximately in a ratio of 1:2 and does not contain appreciable levels of formononetin or biochanin. The outcome of this study was that the ‘high’ isoflavone material resulted in a 2% increase in bone mineral content and density of lumbar spine but had no effect on bone mineral content or density of the femur. The implication from these results is that daidzein and genistein have a modest effect on trabecular bone but no effect on cortical bone.

The literature is somewhat clearer on isoflavones and lipoprotein levels. There are a number of animal and human studies where whole foodstuffs such as soya or other legumes or even relatively crude extracts of soya or other legumes have been fed to recipients and lipoprotein levels monitored. At best these data are highly equivocal and variable. But more importantly, the use of such crude preparations entails the concomitant use of so many plant components including many such as saponins and sterols that are known to have modulating effects on cholesterol metabolism, that it is not possible for even those skilled in the art to draw any relationship between isoflavones and blood lipoprotein levels.

The most telling evidence comes from those studies where supplements are highly enriched for isoflavones and where there no other dietary variation have been used. Three studies using soy extracts enriched for the isoflavones genistein and daidzein have been reported. Two studies failed to find and significant effects of the dietary supplementation on LDL or HDL levels (Nestel et al (1997) “Soy Isoflavones Improve Systemic Arterial Compliance but Not Plasma Lipids in Menopausal and Perimenopausal Women” Arteriosclerosis, Thrombosis and Vascular Biology Biol 17: 3392-3398) and Hodgson et al (1998) “Supplementation with Isoflavonoid Phytoestrogens Does not Alter Serum Lipid Concentrations: A Randomised Controlled Trial in Humans” Journal of Nutrition 128, 728-332). In a third study (Potter S. M. et al as above), 6 months' therapy with a soy powder in hypercholesterolemic, post-menopausal women produced a mean 4.3% increase in HDL levels and a mean 8% decrease in LDL levels. The first two studies would be considered generally to be a more reliable indicator of the lack of effect of soy isoflavones on lipoprotein levels given that the isoflavones were added in a highly concentrated form and necessitated little dietary adjustment. In the third study, the isoflavones were delivered via a soy powder which apart from containing a wide variety of soy components such as saponins and sterols with known cholesterol-modifying properties, also is well known to modify dietary habits through the weight of protein present in the soy product.

U.S. Pat. No. 5,855,892 (Potter) describes a method of altering the concentration of cholesterol constituents in human blood using the isoflavone daidzein. Potter describes the use of soy protein and the isoflavones genistein, daidzein, glycitein and their respective glycosides. A 5.2% increase in HDL-cholesterol concentration was reported in subjects receiving the soy protein/isoflavone composition. It is unclear what the active agent in the compositions is, although the applicants believe it is the soy protein constituent which may be providing the very modest increase in HDL levels.

Further work by Nestel et al (1999 “Isoflavones From Red Clover Improves Systemic Arterial Compliance but Not Plasma Lipids in Menopausal Women” Journal of Clinical Endocrinology and Metabolism 84: 895-898) has shown that dosage with isoflavones from red clover, comprising biochanin, formononetin, daidzein and genistein in the approximate ratio 1.8:1.2:0.2:0.1 also have no effect on plasma lipids. Another study using a similar supplement of isoflavones from red clover and conducted in normocholesteiolemic, premenopausal women found no statistically significant effect of isoflavone supplementation on LDL or HDL levels although there was a slight increase in the HDL₃ sub-fraction (Sanunan S, et al “The effect of supplementation with isoflavones on plasma lipids and oxidisability of low density lipoprotein in premenopausal women” Atherosclerosis 147, 277-283 (1999).

A reasonable summary of the known art would be that isoflavones from the group genistein, daidzein, formononetin and biochanin either singly or in varying combinations have little or no effect on blood lipoprotein levels.

### SUMMARY OF THE INVENTION

It has been surprisingly found by the inventors that compositions comprising high proportions of formononetin relative to one or more isoflavones selected from biochanin, genistein and daidzein, in a therapeutically effective ratio of formononetin to said isoflavones of 15:1 to 2:1, optionally in association with one or more carriers, excipients, auxiliaries and/or diluents, are useful in the prevention and/or treatment of cardiovascular disease, or the beneficial alteration of blood lipoprotein levels, or to reduce the risk of coronary heart disease, or to reduce the risk of arteriosclerosis, or in the beneficial alteration or maintenance of bone density such as to prevent or treat osteoporosis, and/or in the prevention and/or treatment of bone fracture.

These particular health benefits found with a composition containing such a high formononetin content is highly unexpected and surprising for two principal reasons. First, because it is generally believed that any beneficial effect of isoflavones on the cardiovascular system or bone is associated with their estrogenic effect and formononetin displays the weakest estrogenic function of the group of isoflavones comprising genistein, daidzein, formononetin and biochanin. Second, because it also is assumed generally that the human body effectively demethylates formononetin to daidzein, meaning that formononetin should have equivalent function to daidzein.

In accordance with a first aspect of this invention there is provided a composition comprising formononetin and one or more isoflavones selected from biochanin, genistein and daidzein, in a therapeutically effective ratio of formononetin to said isoflavone(s) of 15:1 to 2:1, optionally in association with one or more carriers, excipients, auxiliaries and/or diluents.

Formononetin and one or more isoflavones selected from biochanin, genistein and daidzein are preferably provided in the form of extracts from chickpea, clover, or other plant sources high in formononetin context. The extracts may be prepared by water/organic solvent extracts of legume plants, such isoflavone extractive procedure being well known in the art. Alternatively, isoflavones may be produced by established synthetic techniques as are well known in the art. Formononetin may be in association with one or two or three isoflavones selected from biochanin, genistein and daidzein. Preferably, the formononetin is present in association with biochanin, free of genistein and daidzein or with trace levels or low levels of these components, such as from 0.1% to 5% w/w of isoflavone content.

In accordance with another aspect of this invention there is provided use of formononetin and one or more isoflavones selected from biochanin, genistein and daidzein in the ratio of 15:1 to 2:1 for the manufacture of a medicament for the treatment and/or prevention of cardiovascular disease, or the beneficial alteration of blood lipoprotein levels, or to, reduce the risk of coronary heart disease, or to reduce the risk of arteriosclerosis, or in the beneficial alteration or maintenance of bone density such as in the treatment or prevention of osteoporosis, and/or in the prevention and/or treatment of fracture.

In another aspect of the invention there is provided a method for the prevention and/or treatment of cardiovascular disease, or the beneficial alteration of blood lipoprotein levels, or to reduce the risk of coronary heart disease, or to reduce the risk of arteriosclerosis, or in the beneficial alteration or maintenance of bone density such as in the treatment or prevention osteoporosis, and/or in the prevention and/or treatment of bone fracture, which comprises administering to a human subject a composition comprising formononetin and one or more of biochanin, genistein and daidzein in a therapeutically effective ratio of formononetin to said isoflavones of 15:1 to 2:1, optionally in association with one or more carriers, excipients, auxiliaries, and/or diluents.

### DETAILED DESCRIPTION

Compositions of the present invention comprise formononetin and one or more isoflavones selected from biochanin, genistein and daidzein, in a therapeutically effective ratio of formononetin to said isoflavones of 15:1 to 2:1, optionally in association with one or more carriers, excipients, auxiliaries and/or diluents. Formononetin may be present in association with one or more of biochanin, genistein and daidzein. Where formononetin is in association with a single isoflavone, that isoflavone is preferably biochanin, although having said this, biochanin may be replaced by genistein or daidzein. Where two of biochanin and genistein, biochanin and daidzein or genistein and daidzein are present in addition to formononetin, they may be present in equal amounts on a weight to weight basis, or from 5% through to 95% on a weight to weight basis of a first isoflavone, with a corresponding amount of the second isoflavone. Where the composition comprises formononetin, and biochanin, genistein and daidzein, wherein the ratio of formononetin to said other isoflavones is 15:1 to 2:1, the biochanin, genistein and daidzein may be present in equal amounts on a weight to weight basis, or alternatively in varying amounts, the varying proportions of these isoflavones not being important to the invention. Thus, one “unit” of a combination of biochanin, genistein and daidzein may comprise from 0.1 to 0.99 units biochanin, from 0.1 to 0.99 units daidzein, and from 0.1 to 0.99 units daidzein, giving an “other” (non-formononetin) isoflavone content of one unit. What is particularly significant to the invention is the high formononetin content with regard to other isoflavones, particularly biochanin, genistein and/or daidzein.

The effect of this high formononetin ratio is to produce an unexpectedly large increase in HDL levels in the blood, an effect known to be highly beneficial in protecting against atherosclerosis and coronary heart disease. Similarly, the findings as shown hereafter that the effect of this composition detailed in this invention has a particular and dramatic effect on cortical bone density is both unexpected and indicative of a significant clinical benefit on the initiation and progression of osteoporosis and the resulting risk of bone fracture, particularly of the hip joint, humerus, femur, radius and ulna. The magnitude of these biological effects and resulting clinical outcomes obtained with the composition detailed in this invention is of a magnitude so greater than that known to be obtained with isoflavones generally to indicate that it is a function specifically of this particular isoflavone ratio.

Formononetin and one or more isoflavones selected from biochanin, genistein and daidzein are preferably provided in the form of extracts from chickpea or clover which high in formononetin context. The extracts are preferably water/organic solvent extracts, this isoflavone extractive procedure being well known in the art.

Clover, for example red clover, is a preferred source of formononetin and said other isoflavones. Clovers which may be used include red clover (T. pratense) or subterranean clover (T. subterranean). Many types of red and other forms of clovers are known, and being developed. These legumes may be used in the present invention. The aforementioned isoflavones are preferably prepared by extracting the leguminous material with a water/organic solvent.

Collected plant material may be comminuted or chopped into smaller pieces, partially comminuted or chopped into smaller pieces, or contacted without any pretreatment with generally water and an organic solvent, such as a water miscible organic solvent. The ratio of water to organic solvent may be generally in the range of 1:10 to 10:1 and may for example comprise equal proportions of water and solvent or from 1% to 30% (v/v) organic solvent. Any organic solvent or a mixture of such solvents may be used. The organic solvent may preferably be a C2-10, more preferably a C1-4 organic solvent (such as methanol, chloroform, ethanol, propanol, propylene glycol, erythrite, butanol, butanediol, acetonitrile, ethylene glycol, ethyl acetate, glycidol, glycerol dibydroxyacetone or acetone). The extract in this regard may be prepared by exposing the plant material to the water/solvent mix. Optionally the mixture may include an enzyme which cleaves isoflavone glycosides to the aglycone form. The mixture may be vigorously agitated so as to form an emulsion. The temperature of the mix may range, for example, from an ambient temperature to boiling temperature. Exposure time may be between one hour to several weeks. One convenient extraction period is twenty-four hours at 90° C. The extract may be separated from undissolved plant material and the organic solvent removed, such as by distillation, rotary evaporation, or other standard procedures for solvent removal. The resultant extract containing water soluble and non-water soluble components may be dried to give an isoflavone-containing extract, which may be formulated with one or more pharmaceutically acceptable carriers, excipients and/or auxiliaries.

The extract following distillation contains a small amount of oil which includes isoflavones in their aglycone form (referred to herein as isoflavones). This isoflavone enriched oil, may be subject to HPLC to adjust the isoflavone ratios, or, if at the desired, isoflavone ratio may be dried, for example in the presence of silica, and be formulated with one or more carriers, excipients and/or auxiliaries to give an isoflavone containing extract. Alternatively, isoflavones may be further concentrated by addition to the oil of a non-water soluble organic solvent such as hexane, heptane, octane acetone or a mixture of one or more of such solvents. One example is 80% hexane, 20% acetone w/w having high solubility for oils but low solubility for isoflavones. The oil readily partitions into the organic solvent, and an enriched isoflavone containing extract falls out of solution. The recovered extract may be dried, for example in an oven at 50° C. to about 120° C., and formulated with one or more pharmaceutically acceptable carriers, excipients. and/or auxiliaries. The ratio of isoflavones, from legume extracts, particularly the high content of formononetin to other isoflavones is readily obtained and adjusted, for example by use of clovers of high formononetin content, concentration using various solvents as described above, HPLC fractionation, and the like.

Clover, as the preferred legume source, is readily extract with water/organic solvents. A single source of clover may be used, or a combination of one or more different clovers and/or chickpeas employed.

Formononetin and the other isoflavones referred to herein may be synthetically produced according to methods well known in the art. See for example Kagal et al, Tetrahedron Letters 1962, 593; Mahal et al, J Chem Soc 1934, 1769; Wahala et al, Proc Soc Exp Biol Med 208, 18(1995) at 27-32.

The compositions according to the present invention may include one or more pharmaceutically acceptable carriers. Carriers are selected so as to be acceptable in the sense of being ingredients in the composition and must not be deleterious to the patient. The carriers may be solid or a liquid, or both, and may be formulated with an extract containing the isoflavones at the desired ratios as a unit-dose, for example a tablet, which may contain from 0.5% to 80% by weight of extract or up to 100% by weight-to extract. Compositions may be prepared by any of the well known techniques of pharmacy, for example admixing the extract, optionally including excipients, diluents (for example, water) and auxiliaries as are well known in the pharmaceutical field.

The compositions according to the invention may include one or more agents, such as vitamins (for example, Vitamin A, Vitamin B group, Vitamin C, Vitamin D, Vitamin E and Vitamin K), and minerals (for example, magnesium, iron, zinc, calcium and manganese in the form of pharmaceutically acceptable salts).

The compositions of the invention include those suitable for oral, rectal, optical, buccal (for example, sublingual), parenteral (for example, subcutaneous, intramuscular, intradermal and intravenous) and transdermal administration. The most suitable route in any given case will depend on the nature and severity of the condition being treated and the state of the patient.

Compositions suitable for oral administration may be presented in discrete units, such as capsules, cachets, lozenges, or tablets, each containing a predetermined amount of the extract; as a powder or granules; as a solution or a suspension in an aqueous or non-aqueous liquid; or as an oil-in-water or water-in-oil emulsion. Such compositions may be prepared by any suitable method of pharmacy which includes the step of bringing into association the active compound and one or more suitable carriers (which may contain one or more accessory ingredients as noted above). In general the compositions of the invention are prepared by uniformly and intimately admixing the extract with a liquid or finely divided solid carrier, or both, and then, if necessary, shaping the resulting mixture. For example, a tablet may be prepared by comprising or moulding a powder or granules containing the extract, optionally with one or more accessory ingredients. Compressed tablets may be prepared by compressing in a suitable machine, the extracts in the form of a powder or granules optionally mixed with a binder, lubricant, inert diluents, and/or surface active/dispersing agent(s). Moulded tablets may be made by moulding, in a suitable machine, the powdered compound moistened with an inert liquid binder.

Suitable carriers may be fillers, such as sugars, for example lactose, saccharose, mannitol or sorbitol, cellulose preparations and/or calcium phosphates, for example, tricalcium phosphate or calcium hydrogen phosphate, and also-binders, such as starch pastes using, for example, corn, wheat, rice or potato starch, gelatin, tragacanth, methylcellulose and/or polyvinylpyrrolidone, and, if desired, disintegrators, such as the above-mentioned starches, also carboxymethyl starch, cross linked polyvinylpyrrolidone, agar or algin acid or a salt thereof, such as sodium alginate. Excipients may be flow conditioners and lubricants, for example silicic acid, talc, stearic acid or salts thereof, such as magnesium or calcium stearate, and/or polyethylene glycol. Dragee cores are provided with suitable, optionally enteric, coatings, -there being used, inter alia, concentrated sugar solutions which may comprise gum Arabic, talc, polyvinylpyrrolidone, polyethylene glycol and/or titanium dioxide, or coating solutions in suitable organic solvents or solvent mixtures, or, for the preparation of enteric coatings, solutions of suitable cellulose preparations, such as acetylcellulose phthalate or hydroxypropylmethylcellulose phthalate. Dyes or pigments may be added to the tablets or dragee coatings, for example, for identification purposes or to indicate different doses of active ingredients.

Other orally administrable pharmaceutical compositions are dry-filled capsules made, for example, of gelatine, and soft, sealed capsules made of gelatine and a plasticiser, such as glycerol or sorbitol. The dry-filled capsules may comprise the extracts in the form of granules, for example, in admixture with fillers, such as lactose, binders, such as starches, and/or glicants, such as talc or magnesium stearate, and, where appropriate, stabilisers. In soft capsules, the extract is preferably dissolved or suspended in suitable liquids, such as fatty oils, paraffin oil or liquid polyethylene glycols, to which stabilisers may also be added.

Formulations suitable for buccal (sublingual) administration include lozenges comprising the extracts in a flavoured base, usually sucrose and acacia or tragacanth; and pastilles comprising the compound in an inert base such as gelatine and glycerin or sucrose and acacia.

Compositions of the present invention suitable for parenteral administration conveniently comprise sterile aqueous preparations of the extracts, which preparations are preferably isotonic with the blood of the intended recipient. These preparations are preferably administered intravenously, although administration may also be effected by means of subcutaneous, intramuscular, or intradermal injection. Suitable compositions include water soluble extracts and also suspensions of the active ingredient, such as corresponding oily injection suspensions, there being used suitable lipophilic solvents or vehicles, such as fatty oils, for example sesame oil, or synthetic fatty acid esters, for example ethyl oleate or triglycerides, or aqueous injection suspensions comprising viscosity-increasing substances, for example sodium carboxymethylcellulose, sorbitol and/or dextran, and, where appropriate, also stabilisers. As an example, compositions may conveniently be prepared by admixing the extracts with water or a glycine buffer and rendering the resulting solution sterile and isotonic with the blood. Injectable formulations according to the invention may contain from 0.1% to 60% w/v of the extract and may, for example, be administered at a rate of 0.1 ml/minute/kg.

Formulations suitable for rectal administration are preferably presented as unit dose suppositories. These may be prepared by admixing the extracts with one or more conventional solid carriers, for example cocoa butter, and then shaping the resulting mixture.

Compositions suitable for transdermal administration may be presented as discrete patches adapted to remain in intimate contact with the epidermis of the recipient for a prolonged -period of time. Such patches may contain the extracts in an optionally buffered aqueous solution.

Compositions suitable for transdermal administration may also be delivered by iontophoresis (see, for example, Pharmaceutical Research 3 (6): 318 (1986)) and. typically take the form of an optionally buffered aqueous solution of the extracts. Such compositions may, for example, contain citrate or bis/tris buffer (pH 6) or ethanol/water, with for example 0.05% to 30% w/w extract.

Compositions may be prepared in a manner, and in a form/amount as is conventionally practised. See, for example, Goodman & Gillman, The Pharmacological Basis of Therapeutics (7th Edition, 1985) and Remington's Pharmaceutical Science (Mack Publishing Company, 10th Edition), both of which are incorporated herein by reference. Compositions may contain, for example, from 0.1 mg to 2 g isoflavones, such as 0.1 mg to 200 mg, more particularly 15 mg to 50 mg isoflavones, the ratios on a w/w basis between the isoflavones being as described above.

The compositions of the invention may also be administered to a human in a dietary supplement form. Dietary supplements incorporating the active composition can be prepared by adding the composition to a food in the process of preparing the food. Any food may be used including, but not limited thereto, meats such as ground meats, emulsified meats and marinated meats; beverages such as nutritional beverages, sports beverages, protein fortified beverages, juices, milk, milk alternatives, and weight loss beverages; cheeses such as hard and soft cheeses, cream cheese, and cottage cheese; frozen desserts such as ice cream, ice milk, low fat frozen desserts, and non-dairy frozen desserts; yogurts; soups; puddings; bakery products; salad dressings; and dips and spreads such as mayonnaise, margarine, butter, butter substitute, and other fat containing spreads. The composition is added to the food in an amount selected to deliver a desired dose of the composition to the consumer of the food.

The isoflavones as referred to above may be in the form of a powder, a slurry, in aqueous solution (for example, containing a small amount of oil), particulate form, or dissolved in an organic solvent (such as methanol, ethanol, ethyl acetate or dimethyl sulphoxide).

An effective amount of the compositions of the present invention is administered to a human subject. The actual dosage levels will depend upon a number of factors, such as specific mode of administration, the condition being treated, the condition of the patient and the judgement of the health care giver. Examples of dosages of isoflavones are about 0.1 mg to about 200 mg per day, such as in the order of 1.5 mg/kg (body weight)/day. A convenient dosage form contains about 25 mg to 50mg isoflavone as described herein.

The composition of the present invention comprises formononetin as the principal isoflavone. In the context of this invention, it has been found that the preferred ratio of formononetin to the other three main isoflavones, genistein, daidzein and biochanin embraces the various naturally-occurring forms of isoflavones including their aglycone, glycoside, acetyl or malonyl forms.

In accordance with another aspect of this invention there is provided use of formononetin and one or more isoflavones selected from biochanin, genistein and daidzein in the ratio of 15:1. to 2:1 for the manufacture of a medicament for the prevention and/or treatment of cardiovascular disease, or the beneficial alteration of blood lipoprotein levels or to reduce the risk of coronary heart disease, or in the beneficial alteration or maintenance of bone density such as in the prevention or treatment of osteoporosis, and/or to prevent and/or treat bone fracture. Formononetin and one or more of biochanin, genistein and, daidzein, are provided in a ratio of formononetin to the other isoflavones, whether alone or in combination, in a ratio of 15:1 to 2:1, preferably 10:1 to 5:1. A composition formed therefrom may then be administered to humans.

In another aspect of this invention there is provided a method for the treatment and/or prevention of cardiovascular disease, or the beneficial alteration of blood lipoprotein levels, or to reduce the risk of coronary heart disease, or to reduce the risk of arteriosclerosis, or in the beneficial alteration or maintenance of bone density such as in the treatment or prevention osteoporosis, and/or in the prevention and/or treatment of bone fracture, which comprises administering to a human subject a composition comprising formononetin and one or more of biochanin, genistein and daidzein in a therapeutically effective ratio of formononetin to said isoflavones of 15:1 to. 2:1, optionally in association with one or more carriers, excipients, auxiliaries, and/or diluents.

A further method aspect of this invention is a method for the beneficial alteration of blood lipoprotein levels. In this aspect HDL levels may be increased and/or LDL levels may be decreased. Accordingly there is provided a method of increasing HDL levels in a subject. In another aspect there is provided a method of reducing LDL levels in a subject.

The method aspect of this invention may also extend to a method to decrease the propensity of thrombogenic events in humans.

In a further method aspect of this invention there is provided a method to reduce the risk of vascular disease, coronary heart disease and/or arteriosclerosis in a human.

In a further method aspect of this invention there is provided a method for the beneficial alteration or maintenance of bone density such as in the treatment or prevention of osteoporosis.

In a still further method aspect there is provided a method of preventing and/or treating fractures (including accelerating healing) involving bone with predominant cortical bone tissue, such as those involving the femoral neck, femur, humerus, radius, ulna and tibia.

Each of these above methods involves administering to a human subject a composition comprising formononetin and one or more of biochanin, genistein and daidzein in a therapeutically effective ratio of formononetin to said isoflavones of 15:1 to 2:1, optionally in association with one or more carriers, excipients, auxiliaries and/or diluents. The subjects being treated may be post-menopausal women who are normocholesterolemic or hypercholesterolemic, women who are artherosclerotic, postmenopausal women with low HDL, and males who are hypercholesterolemic or normocholesterolemic, and/or artherosclerotic.

Oral administration of a solid dosage form such as a tablet or capsule is preferred. One or more daily doses is a standard dosing regime. Administration may continue until, for example, lipid levels in the blood are moved to the appropriate levels. However, for maximal benefits on lipid ratios, or prevention of cardiovascular disease, or reduction in the risk of coronary heart disease, or reduction in the risk of arteriosclerosis, or in the beneficial alteration of bone density such as prevention of osteoporosis, and/or in the prevention of bone fractures, administration may be long term, such as for one or more years.

As mentioned above, the ratio of formononetin to other isoflavones as used in the compositions, methods and uses of this invention produces surprising and most advantageous effects in relation to HDL increase (notwithstanding total cholesterol increase), and cortical bone mass increase.

This invention will now be described with reference to the following non-limiting examples.

### EXAMPLE 1

A tablet containing an extract enriched for isoflavones was prepared by methods well known in the fields of pharmaceutical and botanical chemistry. Specifically, the isoflavones are extracted from a legume such as red clover using a standard water/alcohol extract procedure (as described in the patent PCT/AU9800305) and the extract formed into a tablet using standard methods. More specifically, the type of red clover used should contain a mixture of formononetin, biochanin, daidzein and genistein.

Briefly, red clover leaves are harvested and macerated so as to induce enzymatic degradation of isoflavones from their glycosidic form to their aglycosidic form. After standing at ambient temperature for 2 hours, the plant material is snap-frozen by exposure to liquid nitrogen. The material can be stored in this form for up to several years. For extraction, the frozen material is crushed to a fine powder, thawed and placed in a fine gauze bag that is immersed in a solution of 60% ethanol in water. Extraction is carried out at 60° C. for twenty four hours. The supernatant is separated from the undissolved plant material, and the solvent removed by distillation. The aqueous phase containing the isoflavones is extracted again with an organic solvent (either petroleum ether or hexane or acetyl acetate) to remove oils and other polar compounds. The solvent then is removed by distillation and the aqueous phase taken to near-dryness by rotary evaporation. This generates a concentrated extract comprising about 25% isoflavones on a dry weight basis.

This process essentially extracts the isoflavones on a non-preferential basis so that the original ratio of the four isoflavones in the plant is essentially preserved in the final extract. In this example, a strain of red clover was selected that contains the four isoflavones in the approximate ratio (as detected by thin layer chromatography) of 45% biochanin, 40% formononetin, 8% daidzein and 7% genistein.

The dried isoflavone extract was mixed with standard excipients such as methylcellulose to form a 400 mg tablet containing 160 mg clover extract and more specifically, 40 mg of isoflavones comprising 18 mg biochanin, 16 mg formononetin, 3 mg daidzein and 3 mg genistein.

### EXAMPLE 2

A tablet is made according to the procedure detailed in Example 1, but in this case a strain of red clover is selected that has a high formononetin content. The strain selected has a ratio of 82% formononetin, 12% biochanin, 3% daidzein and 3% geristein. After solvent extraction as detailed in Example 1, the dried isoflavone extract has approximately the same isoflavone ratio as in the starting plant material.

A 200 mg tablet is formulated using 100 mg of the dried plant extract containing 25 mg of isoflavones comprising approximately 20 mg formononetin, 3 mg biochanin, 1 mg daidzein and 1 mg genistein.

### EXAMPLE 3

Thirty-six post-menopausal normocholesterolemic women were recruited into a double-blind clinical trial and randomly allotted to one of three treatment arms—(a) placebo tablet, (b) 1× isoflavone tablet daily, or (c) 3× isoflavone tablets daily. The tablets used were those prepared as in Example 1. Treatment continued for 3 months. Blood was collected both at the commencement and completion of the study and analysed for total cholesterol, LDL and HDL levels and clotting factors. No significant changes were found in any of these parameters in any of the treatment arms over the course of the study.

### EXAMPLE 4

Fifty post-menopausal, normocholesterolemic women were recruited into a single-blind clinical trial and randomly allocated to three treatment groups. All three groups received a monthly run-in using a daily placebo-tablet. They then received an isoflavone supplement enriched for formononetin in the form of a tablet as prepared in Example 2. Three doses were used—either 25 mg, 50 mg or 75 mg isoflavones daily. The principal outcomes monitored were total cholesterol, HDL and non-HDL (mainly LDL) levels and bone density of the proximal forearm (predominantly cortical bone) and distal forearm (predominantly trabecular bone). The results are summarised as follows as % change after 6 months' therapy from baseline.

| Isoflavone | Total | | | Apoprotein | Proximal | Distal |
|---|---|---|---|---|---|---|
| concentration | cholesterol | HDL | LDL | B | forearm | forearm |
| 25 mg | 4.85 | 16.5 | 1.18 | −10.59 | 2.9 | −1.4 |
| 50 mg | 6.19 | 28.6 | 6.99 | −9.72 | 4.1 | −1.1 |
| 75 mg | 5.87 | 15.75 | 6.26 | −12.15 | 2.99 | 1.7 |

It can be seen that all three doses of this particular ratio of isoflavones resulted in a variety of statistically significant and clinically significant changes. Total cholesterol levels rose slightly (7%) in all three groups and this was attributable to the dramatic rise in HDL levels. LDL levels were not significantly affected, but HDL levels rose by as much as mean 28% in the 50 mg isoflavone group, an entirely unexpected outcome given the lack of effect on HDL levels observed with other isoflavone studies. A significant decline in blood levels of apoprotein B also was achieved, and again this was entirely unexpected and points to a significant clinical benefit for these women in terms of their risk factors for cardiovascular disease.

Women in the treatment groups, particularly the 50 mg treatment group, also showed a highly significant and positive effect on cortical bone density (proximal forearm) in the first six months (4.1% increase). There was no observed effect on trabecular bone (distal forearm), indicating that this particular isoflavone ratio is having a highly specific effect on cortical bone. Again this is an entirely unexpected outcome given the lack of any previous description of any product that shows specific increase on cortical bone with no effect on trabecular bone.

### EXAMPLE5

Based on the results of Example 4, a 50 mg isoflavone-containing tablet prepared according to Example 2 was tested to see if a particularly refractile treatment group, namely women with high LDL and low HDL could benefit from the treatment of the present invention. This particular grouping of patients are regarded as metabolising cholesterol in a manner different from normocholesterolemic subjects.

The study design was five weeks on the active composition or placebo, and then a cross-over to alternative treatment (active-groups switched to placebo and placebo groups switched to active) for another five weeks. Then all subjects remained on the active for another 12 weeks. Results shown in the table below present data for the “short-active” treatment group which comprise women on active for five weeks, and the data in the “extended-active” column were for women on active for 17 weeks (i.e. 5 plus 12 weeks).

| | Placebo | Short-active | Extended-active |
|---|---|---|---|
| HDL cholesterol (mol) | 1.38 | 1.34 | 1.34 |
| LDL cholesterol (mol) | 5.59 | 5.33 | 5.08 |
| Number of subjects | 22 | 22 | 11 |

This study unexpectedly shows that women with high HDL and low LDL at entry to the study exhibited a significant reduction in LDL, in the order of 10%. This finding correlates with or is somewhat better than current best practice pharmaceutical agents such as the statins. No examples exist in the literature of the use of isoflavones in women with this type of lipoprotein profile. Accordingly, the compounds of the present invention offer treatment for this particularly refractile patient group.

### EXAMPLE 6

25 mg, 50 mg and 75 mg tablets were prepared with the following excipients to form a tablet of total weight 550 mg. The isoflavones were mixed with an acacia gum carrier, then added to a tableting formulation containing mixed tocopherols, cellulose microcrystalline, calcium hydrogen phosphate, soy polysaccharide, magnesium stearate and silica-colloidal anhydrous.

Throughout this specification and the claims which follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

### Patents

PCT Patent No. PCT/AU98/00305 Kelly G. et al: Preparation of isoflavones from legumes.

U.S. Pat. No. 5,855,892 Potter S. M., Henley E. C., Waggle D. H.: Method for decreasing LDL-cholesterol concentration and increasing HDL-cholesterol concentration in the blood to reduce the risk of atherosclerosis and vascular disease.

U.S. Pat. No. 5,506,211 Barnes S. and Blair H. C.: Genistein for use in inhibiting osteoclasts.

U.S. Pat. No. 5,424,331 June 1995 Shylankevich M. 514-456 Pharmaceutical compositions and dietary soybean food products for the prevention of osteoporosis.

### References

Pharmaceutical Research 3(6): 318 (1986)

Anderson, J. J., Ambrose, W. W. and Garner, S. C., (1998) “Biphasic effects of genistein on bone tissue in the ovariectomized, lactating rat model” Proc. Soc. Exp. Biol. Med., March 217(3): 345-50.

Fanti, P., Monier-Faugere M. C., Geng, Z., Schmidt, J., Morris, P. E., Cohen, D., and Malluche, H. H., (1998) “The phytoestrogen genistein reduces bone loss in short-term ovariectomized rats” Osteoporosis Int., 8(3): 274-81.

Goodman Gilman A.(ed.) Goodman & Gilman's The Pharmacological Basis of Therapeutics, 7th Edition, McGraw Hill Publications, 1985

Gordon D. J., Probsfield J. L., Garrison R. J., Neaton J. D., Castelli W. P., Knoke J. D., Jacobs D. R. Jr., Bangdiwala S., Tyroler H. A. (1989) “High-density lipoprotein cholesterol and cardiovascular disease. Four prospective American studies” Circulation 79: 8-15

Hodgson et al (1998) “Supplementation with isoflavonoid phytoestrogens does not alter serum lipid concentrations: a randomised controlled trial in humans” Journal of Nutrition 128: 728-332.

Nestel et al (1997) “Soy isoflavones improve systemic arterial compliance but not plasma lipids in menopausal and perimenopausal women” Arteriosclerosis, Thrombosis and Vascular Biology 17: 3392-3398.

Nestel et al (1999) “Isoflavones from red clover improves systemic arterial compliance but not plasma lipids in menopausal women” Journal of Clinical Endocrinology and Metabolism 84: 895-898.

Potter, S., Baum, J. et al (1998) “Soy protein and isoflavones: their effects on blood lipids and bone density in postmenopausal women” American Journal of Clinical Nutrition, 68(Suppl):1375S-1379S.

Gennaro A. R.(ed.) Remington's Pharmaceutical Sciences Mack Publishing Company, Easton Pa., 17th Edition, 1985.

Samman S., Lyons Wall P. M., Chan G. S. M., Smith S. J., Petocz P. (1999) “The effect of supplementation with isoflavones on plasma lipids and oxidisability of low density lipoprotein in premenopausal women” Atherosclerosis 147: 277-283

1. A composition comprising formononetin and one or more isoflavones selected from biochanin, genistein and daidzein, in a therapeutically effective ratio of formononetin to said isoflavones of 15:1 to 2:1, optionally in association with one or more carriers, excipients, auxiliaries and/or diluents.

2. A composition according to claim 1 wherein the ratio of formononetin to said isoflavones is from 10:1 to 2:1.

3. A composition according to claim 1 comprising formononetin and biochanin.

4. A composition according to claim 1 in the form of a solid dosage unit.

5. A composition according to claim 4 in the form of a tablet, capsule, granular preparation, buccal delivery vehicle or suppository.

6. A composition according to claim 1 wherein said formononetin and biochanin comprise an extract of chickpea or clover.

7. A composition according to claim 6 wherein said clover is selected from red clover (T. pratense), subterranean clover (T. subterranean) or white clover (T. repens).

8. A composition according to claim 7 wherein said clover is red clover.

9. A composition according to claim 1 for the prevention and/or treatment of cardiovascular disease, or the beneficial alteration of blood lipoprotein levels, or to reduce the risk of coronary heart disease, or to reduce the risk of arteriosclerosis, or in the beneficial alteration or maintenance of bone density such as in the prevention or treatment of osteoporosis, and/or in the prevention and/or treatment of bone fracture.

10. A composition according to claim 1 which additionally includes one or more vitamins.

11. A composition according to claim 10 wherein said vitamins are selected from one or more of vitamin C, vitamin D, vitamin E, vitamin K, vitamin A, and vitamin B.

12. A composition according to claim 1 which additionally includes a calcium containing compound (0.5 g to 2 g).

13. A composition according to claim 1 which is incorporate into a beverage.

14. A composition according to claim 13 wherein the beverage is selected from a nutritional beverage, sports beverage, juice, milk or milk alternative.

15. A composition according to claim 1 which is incorporated into a food.

16. A composition according to claim 15 where the food is yogurt, a food bar, a spread, hard cheese, soft cheese, cream cheese or cottage cheese.

17. A composition according to claim 1 in the form of a pharmaceutical composition.

18. A composition according to claim 17 which is in the form of a pill, tablet, capsule, suppository, dragee or sublingual dosage form.

19. Use of formononetin and one or more isoflavones selected from biochanin, genistein and daidzein in the ratio of 15:1 to 2:1 for the manufacture of a medicament for the treatment and/or prevention of cardiovascular disease, or the beneficial alteration of blood lipoprotein levels, or to decrease the propensity of thrombogenic events in humans, or to reduce the risk of coronary heart disease, or to reduce the risk of arteriosclerosis, or in the beneficial alteration or maintenance of bone density such as in the prevention and/or treatment of osteoporosis, and/or in the prevention and/or treatment of fracture.

20. Use according to claim 13, which includes providing formononetin and biochanin in a ratio of 15:1 to 2:1, and forming a composition for administration to humans in admixture with one or more carriers, excipients, auxiliaries or diluents.

21. A method for the treatment and/or prevention of cardiovascular disease, or the beneficial alteration of blood lipoprotein levels, or to reduce the risk of coronary heart disease, or to reduce the risk of arteriosclerosis, or in the beneficial alteration or maintenance of bone density such as in the treatment or prevention osteoporosis, or in the prevention and/or treatment of bone fracture, which comprises administering to a human subject a composition comprising formononetin and one or more of biochanin, genistein and daidzein in a therapeutically effective ratio of formononetin to said isoflavones of 15:1 to 2:1, optionally in association with one or more carriers, excipients, auxiliaries, and/or diluents.

22. A method according to claim 21 which is a method for the beneficial alteration of blood lipid protein levels.

23. A method according to claim 22 which increases the concentration of high density lipoprotein in the blood of said human.

24. A method according to claim 23 which decreases the concentration of low density lipoprotein in the blood of said human.

25. A method according to claim 21 to reduce the risk of vascular disease.

26. A method according to claim 21 to reduce the risk of coronary heart disease.

27. A method according to claim 21 to reduce the risk of arteriosclerosis.

28. A method according to claim 21 for the treatment or prevention of osteoporosis.

29. A method according to claim 21 wherein the ratio of formononetin to said isoflavones is from 10:1 to 2:1.

30. A method according to claim 21 wherein said composition is in the form of a solid dosage form.

31. A method according to claim 21 wherein said dosage form comprises a tablet, capsule, granular preparation, buccal delivery vehicle or suppository.

32. A method according to claim 21 wherein said formononetin and biochanin comprise an extract of chickpea or clover.

33. A method according to claim 32 wherein said clover is selected from red clover (T. pretense), subterranean clover (T. subterranean) or white clover (T. repens).

34. A method according to claim 33 wherein said clover is red clover.

35. A method according to claim 21 wherein the human subject is a pre-menopausal, menopausal or post-menopausal woman.

36. A method according to claim 35 wherein said woman is normocholesterolemic or hypercholesterolemic.

37. A method according to claim 36 wherein said woman is artherosclerotic.

38. A method according to claim 21 wherein said human subject is a normocholesterolemic or hypercholesterolemic male.

39. A method according to claim 38 wherein said male is hypercholesterolemic.

40. A method according to claim 39 wherein said male is normocholesterolemic.

41. A method according to claim 21 which is a method for the prevention or treatment of bone fracture.

42. A method according to claim 41 wherein said method is a method of preventing or treatment fractures involving bone with predominant cortical bone tissue.

43. A method according to claim 42 wherein said bone is selected from the femoral neck, femur, humerus, radius, ulna or tibia bone.
